(19) United States
(12) Patent Application Publication
(10) Pub. No.: US 2016/0095996 A1
(43) Pub. Date: Apr. 7, 2016

(54) NASAL INTERFACE

(71) Applicants: Michael H. Gusky, Weston, FL (US); Nathaniel V. Tran, Lakeville, MN (US); Wayne D. Robens, Lakeville, MN (US); Paul Anthony Pilosi, Minnetonka, MN (US); Jacob Stoesz, Minneapolis, MN (US); Frank Kuhlemann, Baden Wurttemberg (DE); Steven Chance Elliot, Macomb, MI (US); Martin W. Kerber, Eden Praire, MN (US); Martin Baecke, Baden-Wurttemberg (DE); Marcel Borgward, Baden-Wurttemberg (DE)

(72) Inventors: Michael H. Gusky, Weston, FL (US); Nathaniel V. Tran, Lakeville, MN (US); Wayne D. Robens, Lakeville, MN (US); Paul Anthony Pilosi, Minnetonka, MN (US); Jacob Stoesz, Minneapolis, MN (US); Frank Kuhlemann, Baden Wurttemberg (DE); Steven Chance Elliot, Macomb, MI (US); Martin W. Kerber, Eden Praire, MN (US); Martin Baecke, Baden-Wurttemberg (DE); Marcel Borgward, Baden-Wurttemberg (DE)

(73) Assignee: inSleep Technologies, LLC, Weston, FL (US)

(21) Appl. No.: 14/844,890

(22) Filed: Sep. 3, 2015

Related U.S. Application Data
(63) Continuation-in-part of application No. 14/662,798, filed on Mar. 19, 2015.
Application No. 14/662,798 is a continuation of application No. 13/280,650, filed on Oct. 25, 2011, now Pat. No. 8,985,115.
(60) Provisional application No. 61/406,315, filed on Oct. 25, 2010.

(51) Int. Cl. A61M 16/06; A61M 16/08; A61M 16/10; A61M 16/00

(52) U.S. Cl. CPC A61M 16/0666; A61M 16/0057; A61M 16/0875; A61M 16/0816; A61M 16/1045; A61M 2205/42

(57) ABSTRACT

Nasal interface systems and related components. An embodiment of the system employs a nasal interface comprised of a base portion and a soft pad portion. The base portion employs one or more side-entry fluid supply hoses and flow redirection structure which gently and quietly redirects the flow of incoming fluid to avoid the creation of noise. One or more fluid supply hoses are connected to the one or more inlets, preferably by releasable engagement structure. The nasal interfaces of the invention are sleek, unobtrusive, visibly attractive and avoid exposure of expelled gases toward partners of the wearer.

### CROSS-REFERENCE TO RELATED APPLICATIONS

The present application is a continuation-in-part of, and claims priority to, U.S. patent application Ser. No. 14/662,798, filed Mar. 19, 2015 and entitled “Nasal Interface and Removable Pad Therefor,” which itself is a continuation of U.S. patent application Ser. No. 13/280,650, filed Oct. 25, 2011, now U.S. Pat. No. 8,985,115 entitled “Butterfly Nasal Interface,” which itself claims priority to U.S. Provisional Patent Application No. 61/406,315, filed Oct. 25, 2010, the entire disclosures of which are incorporated by reference herein for all purposes.

### TECHNICAL FIELD

The present disclosure generally relates to nasal interfaces for delivering breathing gases to a user, and more particularly relates to nasal interfaces that are small relative to currently existing products on the market, i.e., are sleek in appearance and exhibit a low-profile, but which deliver such gases at high enough flow rates and pressures to treat afflictions such as COPD and asthma with minimal noise.

### BACKGROUND OF THE DISCLOSURE

There are an number of breathing disorders including, but not limited to, snoring, OSA, COPD, and Asthma that could be aided by the use of a supplemental breathing apparatus. Breathing disorders are often treated with the supply of breathable gas or other fluids or inhalants. With the treatment of sleep apnea, continuous positive airway pressure (“CPAP”) machines provide continuous pressure to stent the patient's airway open. However, CPAP machines are often loud and obtrusive. Many patients fail to continue with their therapy merely because their CPAP machines are uncomfortable, unsightly, too noisy, etc.

In order for CPAP machines and other breathable gas delivery systems to work, ideally, a substantially leak proof seal should be maintained between the patient interface and the face of the patient. Forces applied to the patient interface, such as tube drag or the weight of the mask, or components attached to the mask, tend to disrupt the seal formed between the patient interface and the patient.

There have been many designs to create a substantially leak proof seal in order to provide breathable gas to a patient. Various solutions have been proposed for creating a substantially leak proof seal while reducing the undesirable forces that may be applied to a mask or headgear.

One solution which has been proposed to reduce the application of undesirable forces on the patient interface where a trunk-type tube is used to supply air to the interface is a headgear to provide stability to the patient interface and maintain the seal during the application of the forces, including tube drag. The headgear assembly may be designed such that stabilizing straps are provided at an angle with respect to the patient interface and the face of the patient to counteract the undesirable forces. In one known mask assembly, the headgear includes a cap portion with four straps. In use, the cap portion engages the back of the patient's head and two lower straps extend between the cap portion and a nasal mask while the two upper straps extend between the cap portion and a forehead support. Such headgear assemblies, however, are typically too weak to be able to resist the substantial dislodging forces imposed on the interface by the trunk hose.

Not only is the trunk hose and headgear assembly of the above solution unpleasant to the eye, but the size and location of the hose overly restricts the patient's movement by applying unnecessary forces to the patient's head. The headgear is very uncomfortable to the patient, as well as those that may be sleeping near the patient.

In addition to the uncomfortable nature of the patient interfaces, the machines also create a lot of noise. The flow of air through the machine often creates areas of turbulence, and hence noise and vibration.

Also, the size of the nasal interfaces presently in existence are dictated by often competing ergonomic and functional considerations. It is commonly understood that the smaller the individual components of a CPAP system are, the less undesirable the system will be to use, which will drive more compliance. Conversely, however, the smaller the components of the system, the less effective the system is likely to be, due to the decreased sizes of flow pathways and concomitant flow rate reductions and noise increases.

Thus, it is an object of one aspect of the present invention to provide sleek, comfortable, and quiet patient interfaces that provide the least resistance to the patient's movement while still maintaining a substantially leak proof seal between the patient interfaces and the face of the patient.

It is also an object of one aspect of this invention to provide CPAP systems having relatively small components, but which permit the necessary flow rates and pressures in a quiet manner.

It is also an object of one aspect of this invention to provide bases for patient interfaces which have internal airflow-directing features to reduce turbulence, and the sound/noise which accompanies such turbulence.

It is also an object of one aspect of this invention to provide bases for patient interfaces which are manufactured in two or more parts for ease of manufacture.

It is also an object of one aspect of this invention to provide bases for patient interfaces having one or more annular spaces/channels into which may be fit corresponding attachment structure of a nasal interface pillow.

It is also an object of one aspect of this invention to provide bases for patient interfaces having one or more sound deadening features.

It is also an object of one aspect of this invention to provide bases for patient interfaces having one or more humidification features.

It is also an object of one aspect of this invention to provide bases for patient interfaces having quick-disconnect hose connectors to permit easy replacement or cleaning of the interface.

These and other objects are achieved by the structure and methods of use disclosed herein.

### SUMMARY OF THE DISCLOSURE

A nasal interface system and related components. An embodiment of the system employs a nasal interface comprised of a base portion and a soft pad portion. The base portion employs one or more side-entry fluid supply hoses and flow redirection structure which gently and quietly redirects the flow of incoming fluid to avoid the creation of noise. The one or more fluid supply hoses are connected to the one or more inlets, preferably by releasable engagement structure. The nasal interfaces of the invention are sleek, unobtrusive, visibly attractive and avoid exposure of expelled gases toward partners of the wearer.

In accordance with one aspect of the invention, nasal interfaces that altogether avoid the drag associated with forward facing hoses are disclosed.

In accordance with another aspect of the invention, nasal interfaces that provide a comfortable alternative to other nasal interfaces are disclosed.

In accordance with a further aspect of the invention, nasal interfaces having a substantially leak proof seal between the nasal interface and the patient are disclosed.

In accordance with an even further aspect of the invention, nasal interfaces which reduce the noise and vibrations associated with prior patient interfaces is disclosed.

In accordance with yet another aspect of the invention, nasal interfaces that provide very little resistance to a patient's movement are disclosed.

In accordance with another aspect of the invention, CPAP systems having relatively small components, but which permit the necessary flow rates and pressures to treat ailments such as COPD and asthma in a quiet manner, are disclosed.

In accordance with another aspect of the invention, bases for patient interfaces which have internal airflow-directing features to reduce turbulence, and the sound/noise which accompanies such turbulence are disclosed.

In accordance with another aspect of the invention, bases for patient interfaces which are manufactured in two or more parts for ease of manufacture are disclosed.

In accordance with another aspect of the invention, bases for patient interfaces having one or more annular spaces/channels into which may be fit corresponding attachment structure of a nasal interface pillow are disclosed.

In accordance with another aspect of the invention, bases for patient interfaces having one or more sound deadening features are disclosed.

In accordance with another aspect of the invention, bases for patient interfaces having one or more humidification features are disclosed.

In accordance with another aspect of the invention, bases for patient interfaces having quick-disconnect hose connectors to permit easy replacement or cleaning of the interfaces are disclosed.

The details of one or more implementations are set forth in the accompanying drawings and the description below. Other features will become apparent from the description, the drawings, and the claims. Further features and advantages of the invention, as well as structure and operation of various embodiments of the invention, are disclosed in detail below will reference to the accompanying drawings.

### BRIEF DESCRIPTION OF THE SEVERAL VIEWS OF THE DRAWINGS

FIG. 1 is a top perspective view of one half of a base of a nasal interface in accordance with one aspect of the present invention.

FIG. 2A is a top rear perspective exploded view of a nasal interface and air delivery hose system employing the embodiment of the nasal interface base shown in FIG. 1.

FIG. 2B is a cross-sectional elevational view of an air supply hose suited for use with embodiments of invention.

FIG. 2C is a cross-sectional elevational view of an alternative air supply hose suited for use with embodiments of the invention.

FIG. 3 is a top rear right perspective view of the assembled nasal interface and air delivery hose system of FIG. 2.

FIG. 4 is a front elevational view of the assembled nasal interface of FIG. 3.

FIG. 5 is a right side elevational view of the assembled nasal interface of FIG. 3, which is a mirror image of the left side elevational view thereof.

FIG. 6 is a top plan view of the of the assembled nasal interface and air delivery hose system of FIG. 3.

FIG. 7 is a bottom plan view of the assembled nasal interface and air delivery hose system of FIG. 3.

FIG. 8A is a front cross-sectional elevational view of the assembled nasal interface and air delivery hose system of FIG. 3 taken along lines 8A-8A of FIG. 6 showing inspiration airflow through the interface.

FIG. 8B is a front cross-sectional elevational view of the assembled nasal interface and air delivery hose system of FIG. 3 taken along lines 8A-8A of FIG. 6 showing expirational airflow through the interface.

FIG. 9A is a front cross-sectional elevational view of an alternate nasal interface base.

FIG. 9B is a front cross-sectional elevational view of a further alternate nasal interface base.

FIG. 9C is a front cross-sectional elevational view of a further alternate nasal interface base.

FIG. 9D is a front cross-sectional elevational view of a further alternate nasal interface base.

FIG. 9E is a front cross-sectional elevational view of a further alternate nasal interface base.

FIG. 9F is a front cross-sectional elevational view of a further alternate nasal interface base.

FIG. 9G is a front cross-sectional elevational view of a further alternate nasal interface base.

FIG. 9H is a front cross-sectional elevational view of a further alternate nasal interface base.

FIG. 9I is a front cross-sectional elevational view of a further alternate nasal interface base.

FIG. 9J is a front cross-sectional elevational view of a further alternate nasal interface base.

FIG. 9K is a front cross-sectional elevational view of a further alternate nasal interface base.

FIG. 9L is a front cross-sectional elevational view of a further alternate nasal interface base.

FIG. 9M is a front cross-sectional elevational view of a nasal interface base.

FIG. 9N is a front cross-sectional elevational view of a further alternate nasal interface base.

FIG. 10 is a top perspective view of one half of a still further alternative embodiment of a nasal interface in accordance with the present invention.

FIG. 11 is a top rear perspective exploded view of a nasal interface and air delivery hose system employing the embodiment of the nasal interface base shown in FIG. 10.

FIG. 12 is a top rear right perspective view of the assembled nasal interface and air delivery hose system of FIG. 11.

FIG. 13 is a front elevational view of the assembled nasal interface and air delivery hose system of FIG. 12.

FIG. 14 is a right side elevational view of the assembled nasal interface and air delivery hose system of FIG. 12.

FIG. 15 is a top plan view of the of the assembled nasal interface and air delivery hose system of FIG. 12.

FIG. 16 is a bottom plan view of the assembled nasal interface and air delivery hose system of FIG. 12.

FIG. 17A is a front cross-sectional elevational view of the assembled nasal interface and air delivery hose system of FIG. 12 taken along lines 17-17 of FIG. 15 showing inspiration airflow through the interface.

FIG. 17B is a front cross-sectional elevational view of the assembled nasal interface and air delivery hose system of FIG. 12 taken along lines 17-17 of FIG. 15 showing expiration airflow through the interface.

FIG. 18 is a front cross-sectional elevational view of an assembled nasal interface and air delivery hose system showing a humidification element with handling recesses.

FIG. 19 is a top perspective view of one half of a still further alternative embodiment of a nasal interface in accordance with the present invention.

FIG. 20 is a top rear perspective exploded view of a nasal interface and air delivery hose system employing the embodiment of the nasal interface base shown in FIG. 19.

FIG. 21 is a top rear right perspective view of the assembled nasal interface and air delivery hose system of FIG. 19.

### DETAILED DESCRIPTION OF PREFERRED AND ALTERNATIVE EMBODIMENTS

Before describing in detail exemplary embodiments that are deemed to within the scope of the present inventions, it should be observed that the inventions disclosed herein reside primarily in combinations of the various features and embodiments of a breathing apparatus and/or elements of a breathing apparatus, although certain individual features of such apparatus are considered to be within the scope of one or more of the inventions as well.

Accordingly, the apparatus components have been represented where appropriate by conventional symbols in the drawings, showing only those specific details that are pertinent to understanding the embodiments of the present inventions so as not to obscure the disclosure with details that will be readily apparent to those of ordinary skill in the art having the benefit of the description herein.

In this document, relational terms, such as “first” and “second,” “top” and “bottom,” and the like, may be used solely to distinguish one entity or element from another entity or element without necessarily requiring or implying any physical or logical relationship or order between such entities or elements.

The terms “comprises,” “comprising,” “comprise” or any other variation thereof are intended to cover a non-exclusive inclusion, such that a process, method, article, or apparatus that comprises a list of elements does not include only those elements, but may include other elements not expressly listed or inherent to such process, method, article, or apparatus.

The term “plurality of” as used in connection with any object or action means two or more of such object or action.

A claim element proceeded by the article “a” or “an” does not, without more constraints, preclude the existence of additional identical elements in the process, method, article, or apparatus that includes the element.

Terms such as “air”, “fluid” and “breathable gas” are interchangeable and can refer to any fluid such as breathable air, oxygen, anesthesia, inhalants, etc.

Terms such as “naris,” “nares,” “nose” or “nostrils” may be used interchangeably.

Generally, one aspect of present disclosure is a nasal interface through which fluid is quietly but efficiently supplied to a patient's nose.

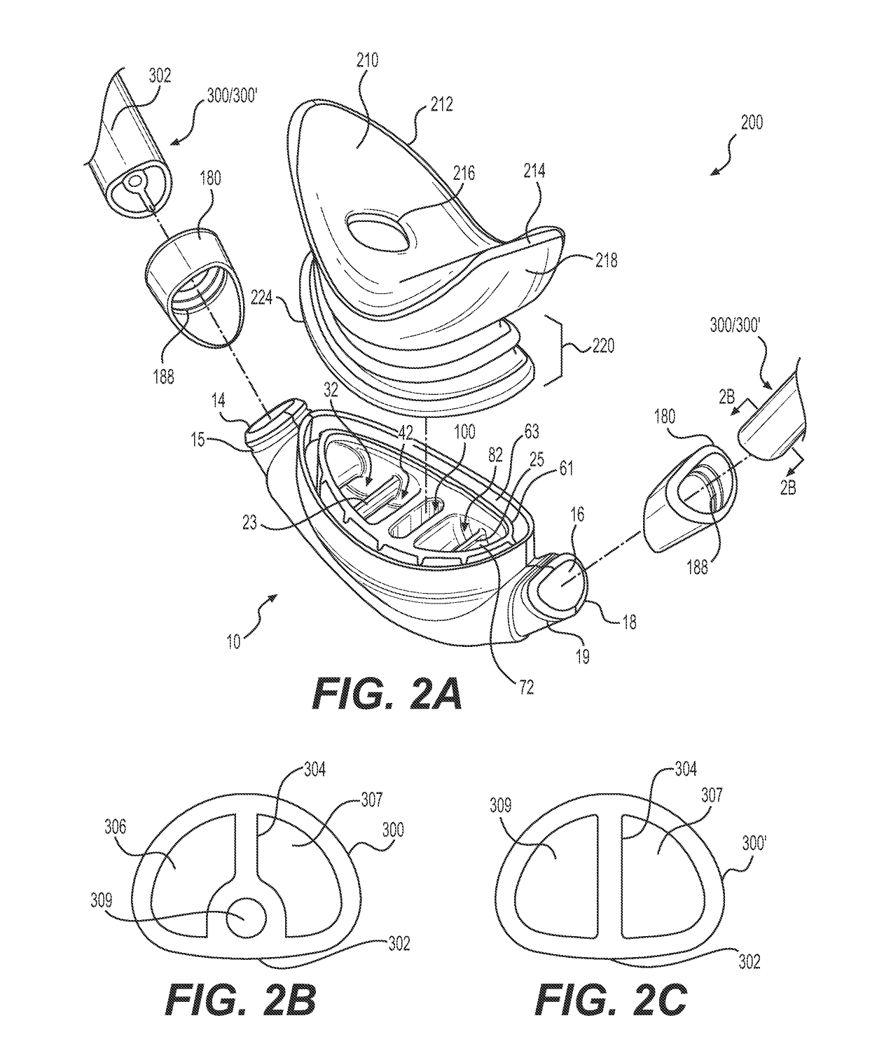

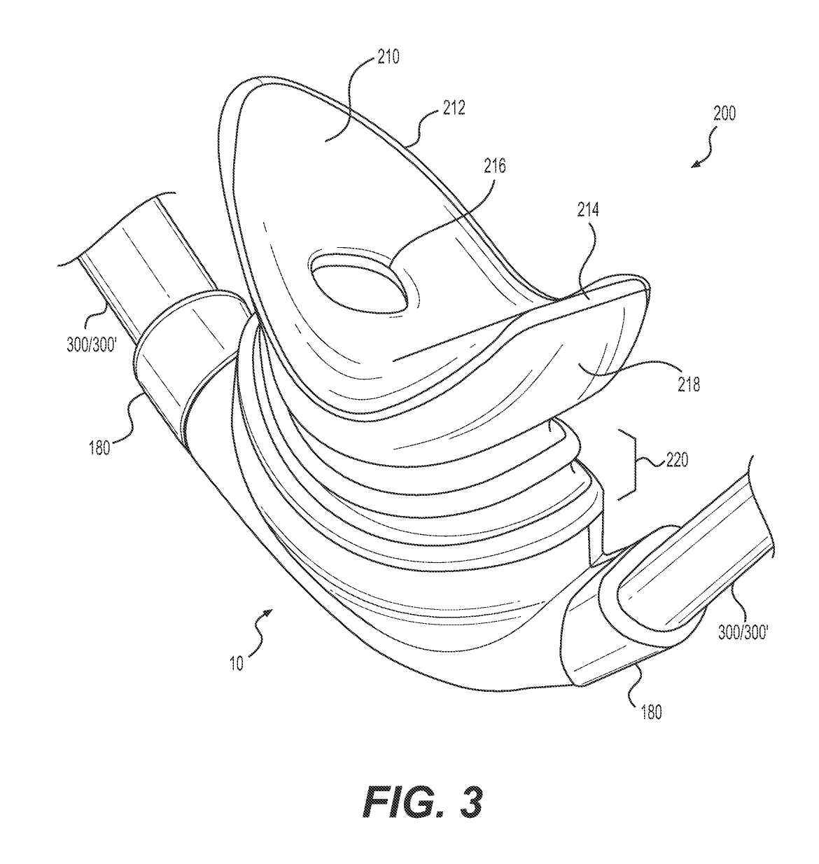

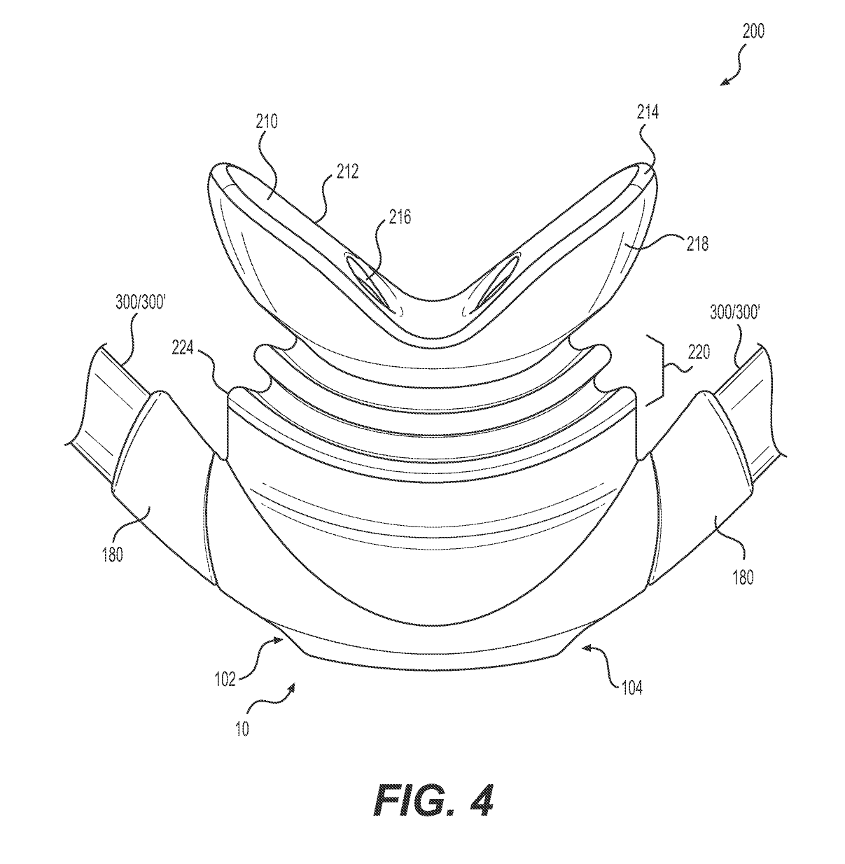

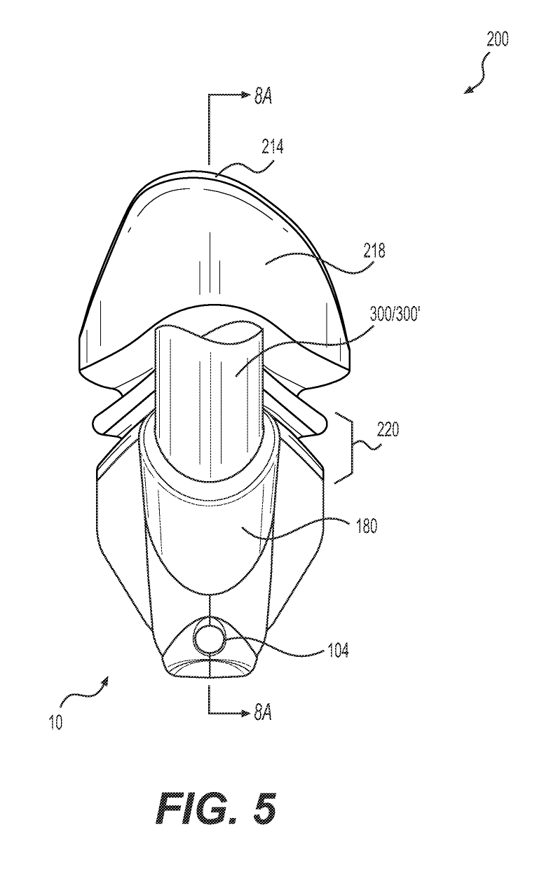

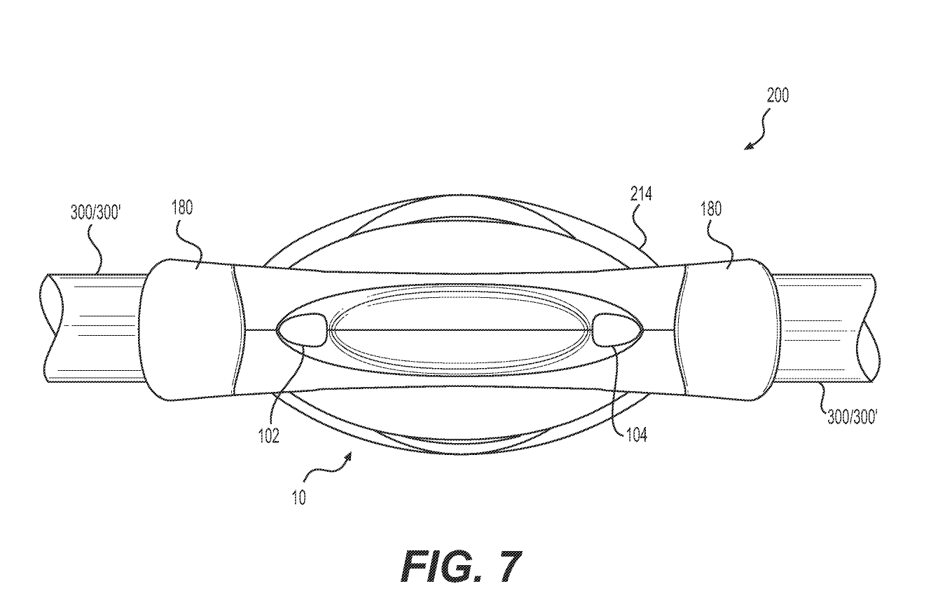

Referring to FIGS. 2A-8B, a first illustrative exemplary embodiment of a nasal interface includes a hard base portion 10 and a soft pad portion 200. The soft pad 200 can comprise a silicon pad, and the base portion 10 can be relatively harder than the soft pad portion 200. The soft pad portion 200 is preferably unitary in nature and includes a winged shaped structure with wings 218. The wings 218 include upper surfaces, wall(s) or web(s) 210 with respective distal ends 214. The wings 218 respectively include therein one or more airflow holes or apertures 216. These air holes 216 may be sized, positioned, and physically separated (spaced apart) on the upper surfaces 210 of wings 218 to locate the air holes in or near registry with a user's nostrils. The upper surfaces 210 of the wings 218 engage the underside of a user's nose. As also described below, this allows air from the nasal interface to flow to the user's nostrils. In other embodiments, the upper surfaces 210 could include protrusions or other physically noticeable features about or near the air holes 216 to aid in positioning of the nasal interface with respect to a user's nostrils.

In an illustrative embodiment, the upper surfaces 210 may be positioned relative to each other at an angle α of about 90° on the wings 218 of a nasal interface in accordance with the present disclosure. The selection of the angle α is based upon ergonomic studies. Of course, in various embodiments the included angle α may vary from about a 180° included angle to about a 45° included angle. The upper surfaces 210 may seal around the users nostrils. The upper surfaces 210 may be pressed smoothly against the user's nose by the system's pressure (e.g., mechanical pressure of the upper surfaces 210 of wings 218 of the nasal pad 200 as held against the underside of a user's nose) to form this seal.

The upper surfaces 210 of wings 218 slope across a width of the soft pad 200 from a relatively higher position to a relatively lower position. While not necessary to practicing the invention, this slope allows a nasal interface in accordance with the present disclosure to be positioned on a user's nose to conveniently accept tubing.

The soft pad portion 200 includes a lower border 224. In the illustrative embodiment, the lower border 224 aids in coupling the soft pad portion 200 to the base portion 10. The coupling can be any convenient coupling, such as a mechanical connection, an adhesive, welding, etc. In some embodiments, the lower border 224 may have a more or less round or oval cross section. The lower border 224 may be tensioned over an upper border of the base portion 10, which can be formed to fit using, for example, a key-lock type connection. The curved shape may facilitate a tight sealing between the base and the soft pad 200 because of the tensile forces working radially toward to the center. Depending upon the use of the nasal interface and the type of coupling, the base portion 10 can be removable from the soft pad portion 200. Such removal facilitates cleaning the nasal interface or changing one of the base portion 10 or the soft pad portion 200.

The soft pad portion 200 may optionally include a bellows 220. Bellows 220 may provide flexibility in height of the nasal interface and the angle at which the upper surfaces 210 reside with respect to a user's nostrils. The bellows 220 can function as a spring if there is pressure between the user's nose and the base portion 10.

The illustrated bellows 220 includes vertical parts (e.g., relative to the base) and horizontal parts (e.g., relative to the base). The vertical parts may impart stability to the shape. The horizontal parts may allow a vertical movement of upper surfaces 210. This vertical movement may be for the whole soft pad portion 200, relative to the base portion 10. The vertical movement may be on one side of the pad, or both sides of the pad (including movement to different degrees on different sides). Thus, the bellows 220 allows the angle of the upper surfaces 210 relative to the base to change. This flexibility of the bellows and ability for the angle of the upper surfaces 210 to change may result in a self-sealing effect between a user's nose and the upper surfaces 210. While illustrated as extending around the nasal interface, the bellows 220 may extend only around a portion of the nasal interface.

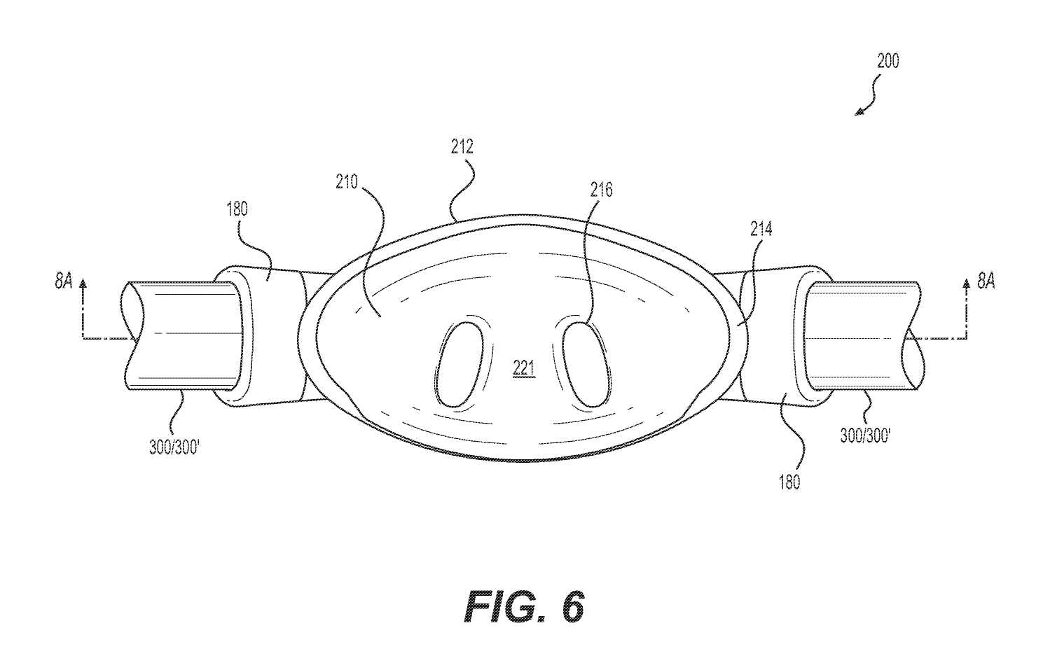

The holes 216 in the upper surfaces 210 let air into and out of a user's nostrils. The size and shape of these holes can be adjusted depending upon the desired flow and acoustic characteristics of a given design in accordance with the disclosure. A single opening (not shown) intended to be co-extensive with the position of both of a typical user's nostrils may also be used. The hole or holes 216 may have a cross section optimized for low pressure losses and low noise from the airflow through the holes. Also, in the illustrated embodiment, the holes 216 are near the edges between upper surfaces 210, and side surface 212. This position is shown in the illustrative embodiment because most people have nostrils beginning very near or directly above the user's upper lips. As further shown in the illustrated embodiment, the wing portions 218 and their upper surfaces 210 are interconnected by way of a central connecting portion 221, which is interposed between the air holes 216 of the wings 218 and can seal against the central bottom strip (columella) of the user's nose during use. In the embodiment illustrated in FIG. 6, the connecting portion 221 of the pad 10 is devoid of any aperture.

Because a user's nostrils can begin directly from the upper lip there may not be a significant surface to seal the upper surfaces 210 to the user's nose. To address this, an illustrative embodiment may include side surface 212. The side surface 212 can aid in providing a seal between the nasal interface and the user. It can do so by the side surface 212 abutting and pressing against one or more of the user's upper lip, philtrum and/or columella.

The nasal interface may be positioned at an angle against the nose and upper lip of a user. In such an embodiment, the surfaces of the soft pad portion 200 assume a very soft and safe position on the user. Because of the flexibility of the soft pad 200, such as provided by the bellows 220, the nasal interface can be adjusted relative to three planes: with respect to the upper lip, and the two wings 218 with respect to a user's nostrils. This flexibility in positioning allows the position of the nasal interface to be highly self-adjusting. Gliding of the nasal interface may be reduced and/or prevented by this structural form. In addition tilting of the base portion 10 may prevent leakage due to the spring like function of the bellows 220 and the pressure the bellows 220 provides against a user's nose and the flexibility in angle of the upper surfaces 210 that the bellows 220 permits. Very low forces are needed with embodiments in accordance with the disclosure to effectively seal the nasal interface with a user's nostrils.

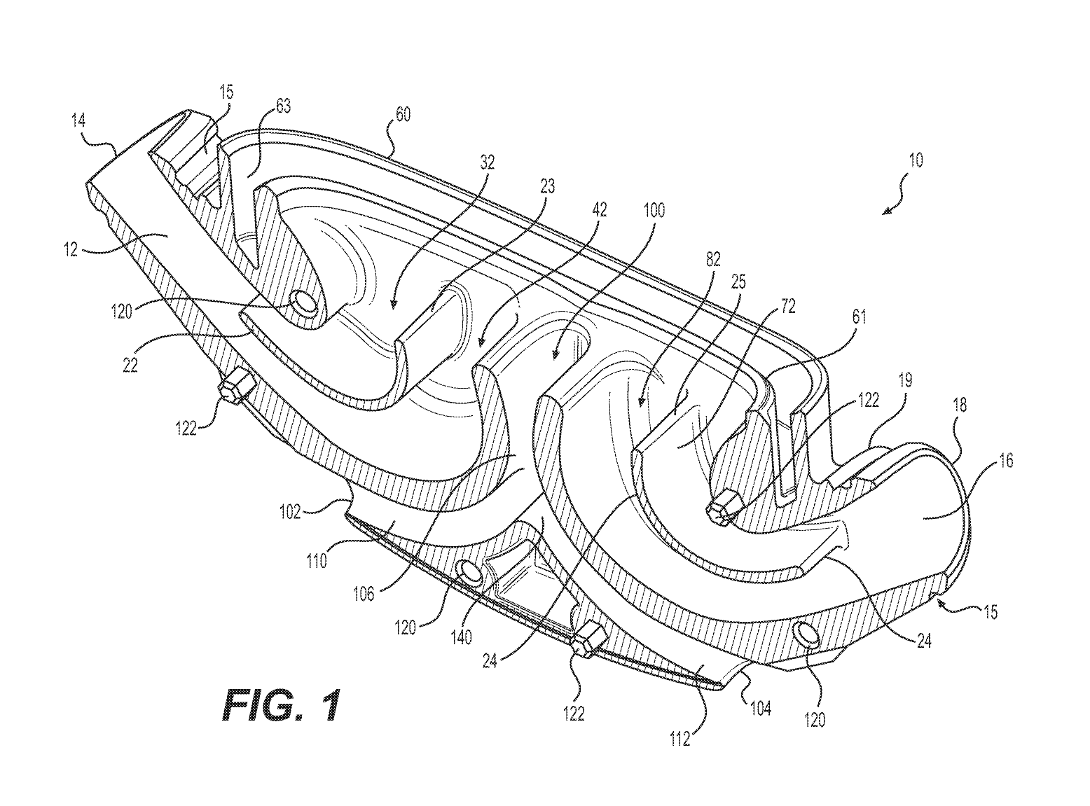

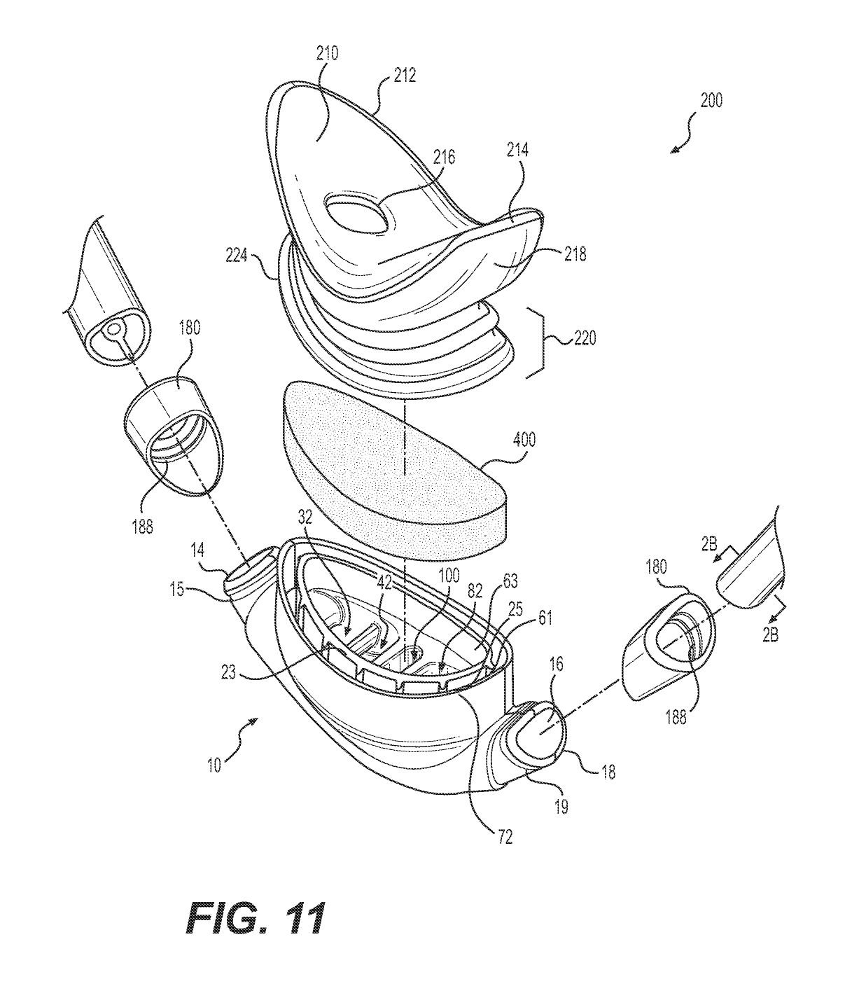

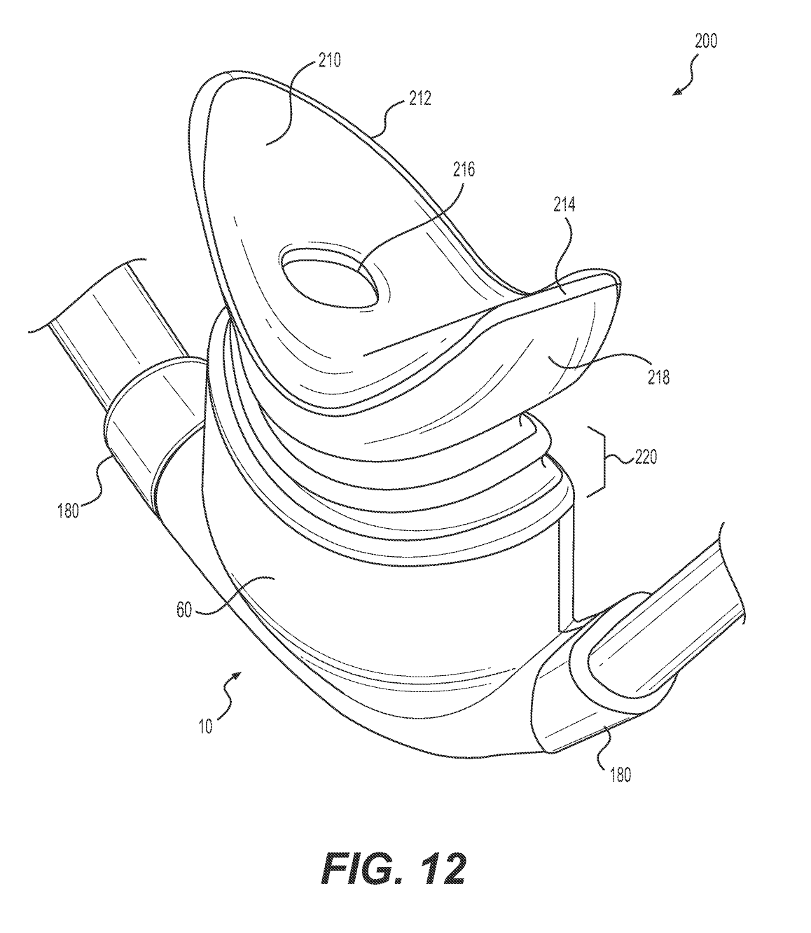

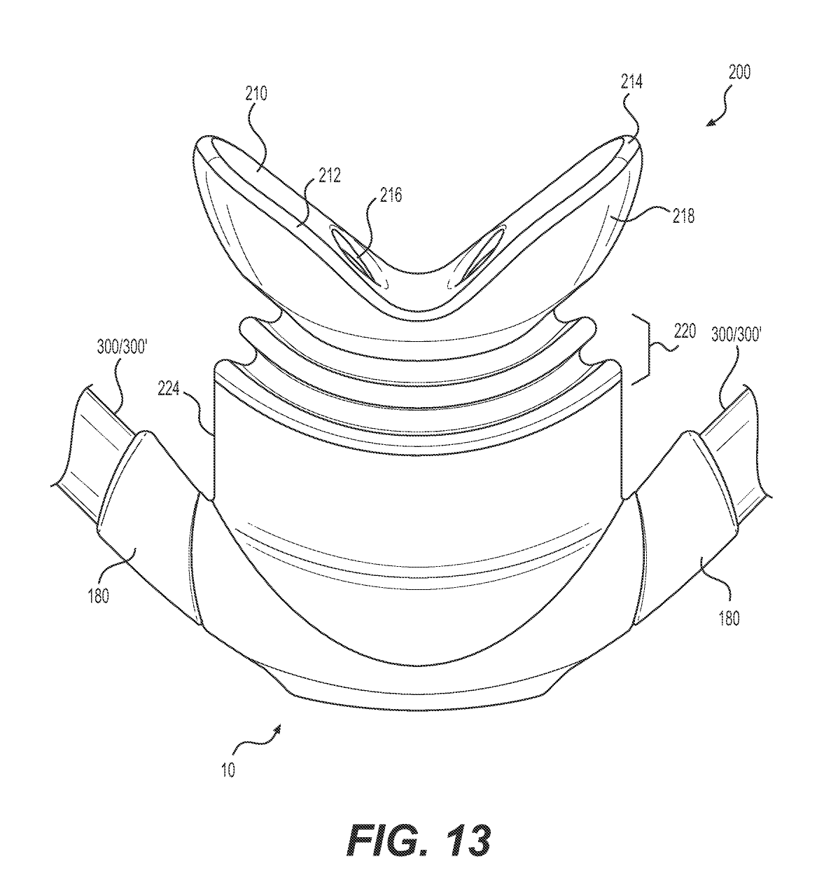

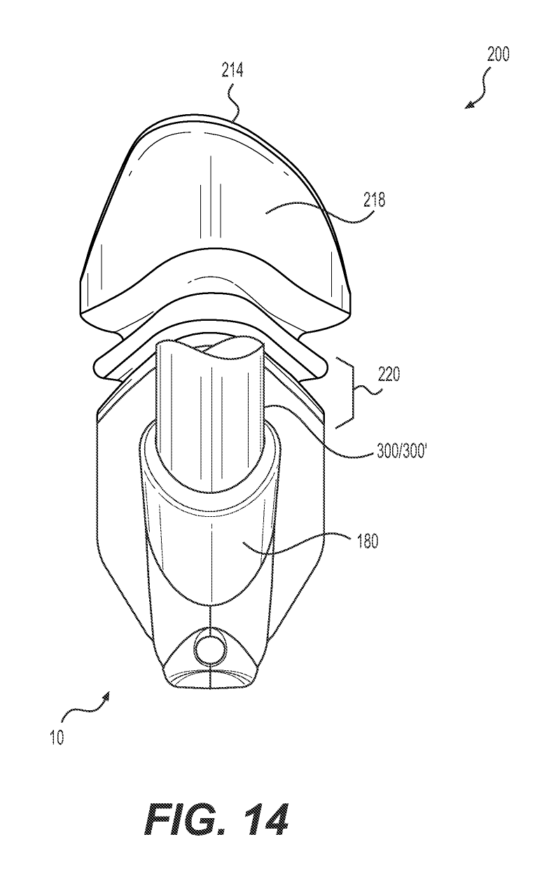

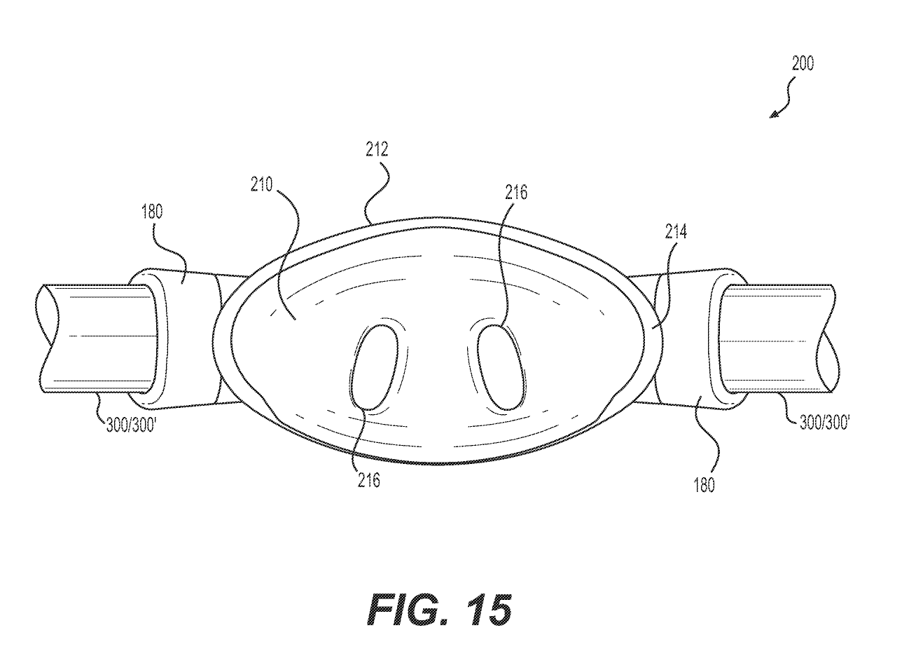

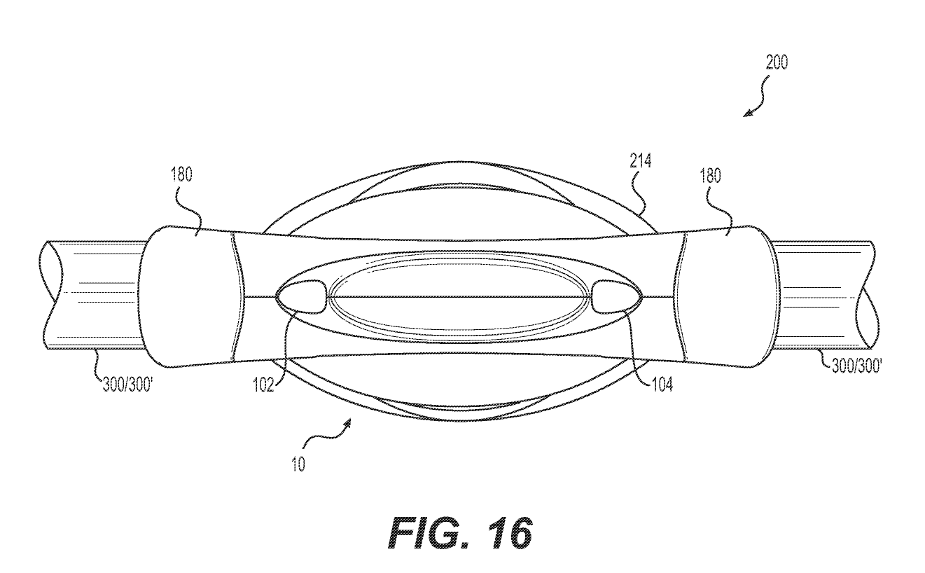

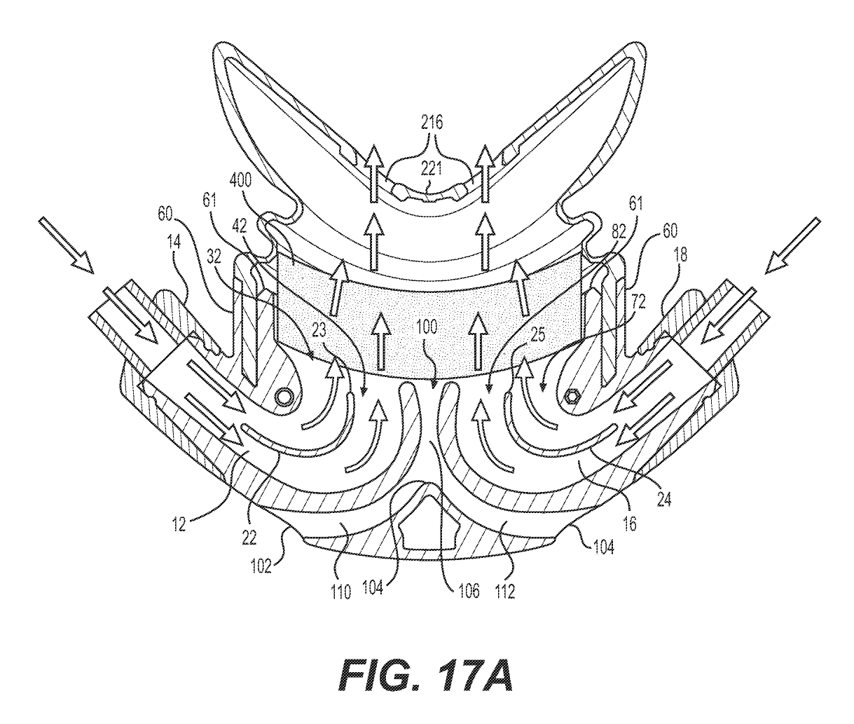

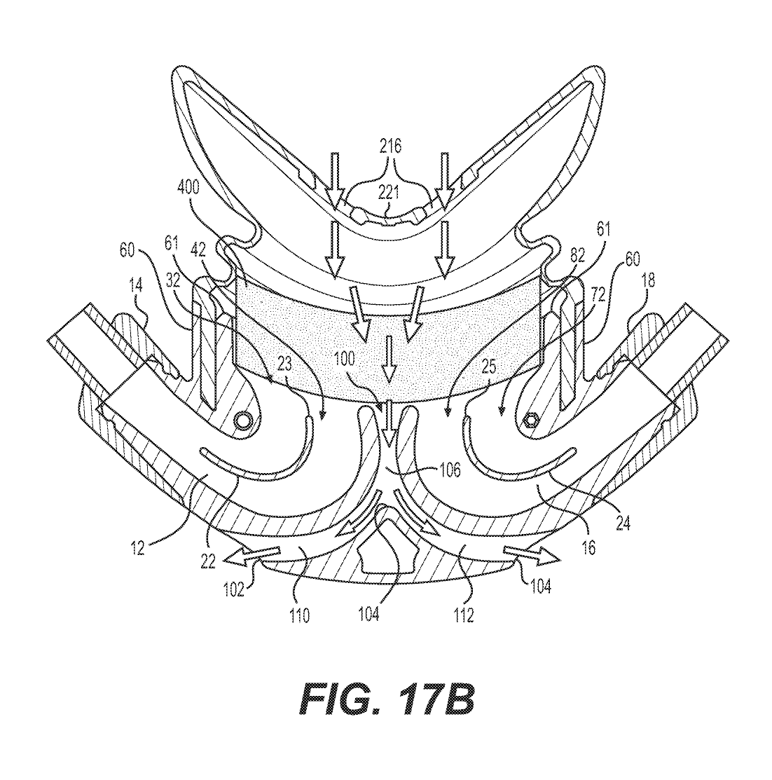

In the illustrated embodiment, the base portion 10 can be connected (e.g., glued, welded, snap or press fit, etc.) with one or more fluid supply hoses, such as hoses 300/300′ cross-sections of which are shown in FIGS. 2B and 2C, or any fluid supply hoses. The illustrative embodiments shown in FIGS. 1-17 utilize two hoses, rather than one hose. The reference to “hose 300” or “hose 300′,” or to any hose connected or connectable to a nasal interface base in accordance with the inventions disclosed herein, is considered to encompass any fluid supply hose of any shape, manufacture, material or other properties.

The hoses 300 connect to inlets 14, 18 defined by the base 10. However, a system in accordance with this invention may be configured with a single hose, or any number of hoses.

The inlets 14, 18 can be oriented at different angles. In one embodiment, the inlets 14, 18 can be adjusted in every direction that is reasonable to provide a good seal with the nasal interface. While the illustrated embodiments depict two inlets 14, 18 for connecting to two hoses 300 on either side of the nasal interface, other arrangements may also be utilized. For example, one or more hoses may be connected at various locations on the nasal interface, such as the front of the nasal interface, or other location.

The hoses 300, in addition to supplying air to the nasal interface, may be utilized for holding the mask in the right position for maintaining connection to (e.g., sealing engagement with) the user's nose. In one embodiment, the hoses may come from a direction above the user's ear. In other embodiments, a headgear arrangement not including the hoses 300 (and/or in addition to the hoses) may be utilized for positioning the nasal interface relative to the user.

The base portion 10 may define one or more airflow vent passages or outlet(s) such as exhaust passages 110, 112 ending with respective openings 102, 104. Openings 102, 104 fluidly communicate the interior of nasal pillow 200 with the ambient via passages 110, 112. The size and shape of the vent outlet(s) can be selected in accordance with the flow and acoustic characteristics of a given design. The vent outlet(s) 102, 104 can be, as shown in the illustrative embodiments, open to the ambient. In one embodiment, the vent outlet(s) may be designed to allow airflow of about 20 l/min at 2 mbar overpressure inside the mask. This may reduce and/or prevent a CO₂-concentration to the user in the nasal interface from being too high. Additionally, the vent outlet(s) 102, 104 may be sized to provide an open breathing interface, such that the user may generally breathe ambient air entering through the outlets if the CPAP blower ceases to operate. The ambient air may be supplemented with gases (e.g., oxygen, etc.) supplied via the hose(s) 300. An additional tube or lumen 306 can be used to fluidly communicate the interior of base 10 and/or nasal pillow 200 with a measurement transducer to measure the pressure within the nasal interface. It can also be used for pressure feedback to the air supply. The hoses may also contain multiple lumens where one of the lumens can provide pressure feedback to the air supply.

For a nasal interface which fits a wide range of users, the nasal interface can have a relatively small dimension with respect to users' noses. In addition, the shape of the wings 218 might be increased or decreased depending upon the target size nose of users. The base portion 10 preferably has a relatively small, sleek profile and appearance. The inner cross section allows its overall shape to be highly stylized. The structure of the disclosed illustrative embodiments allows the base portion 10 to move sideward and upwards with reduced displacement of wings 218 at the user's nose.

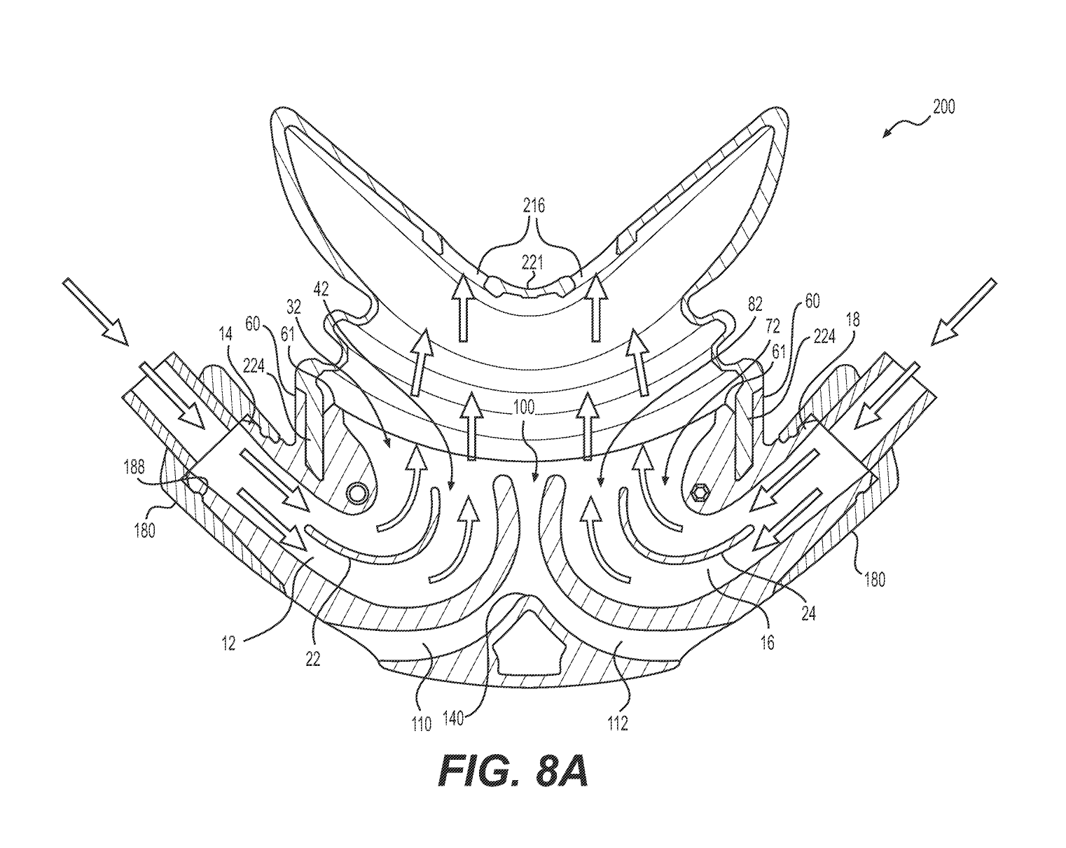

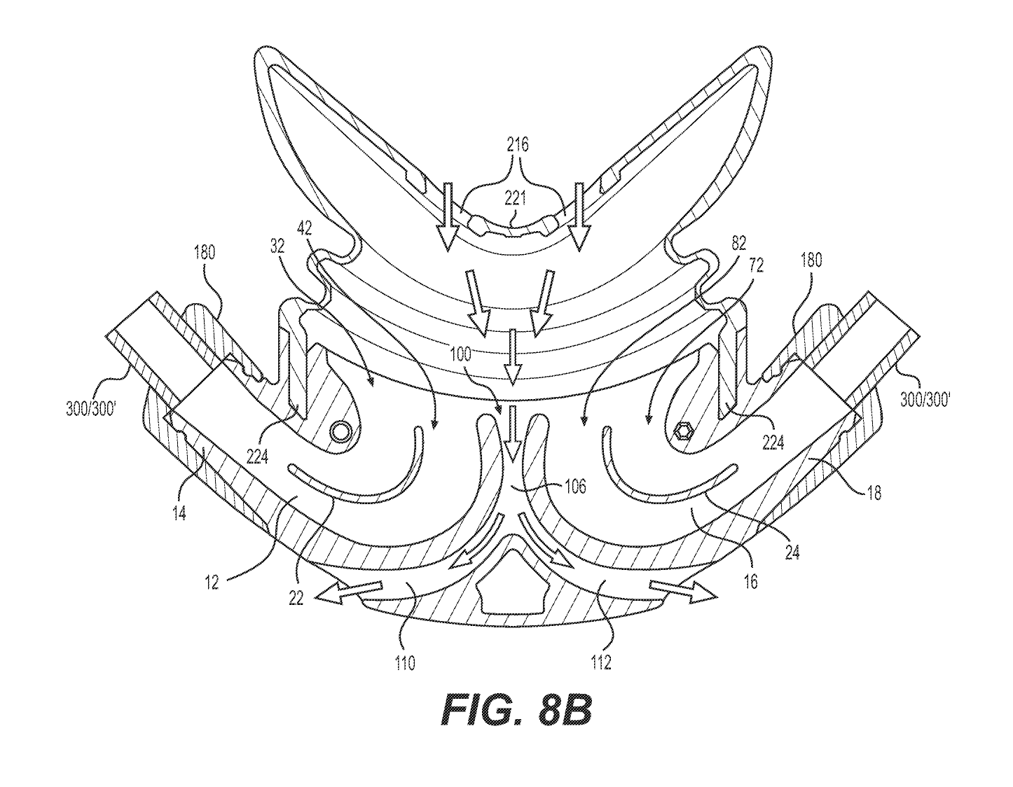

The inside of one half of a base 10 in accordance with one embodiment of one aspect of the invention is depicted in FIGS. 1 and 8A. This half-base can be separately manufactured and connected to an identical half-base 10 (i.e., mirror-image) to form a fully operational base such as base 10 shown in FIGS. 1-8B. Connection lugs 122 and corresponding lug-receiving sockets 120 may be added to assist in locating and securing the two base halves together. The halves may be connected by inserting male lugs 122 into corresponding female sockets 120. However, base 10 can be formed as one piece, or as separate pieces with or without mating members. Furthermore, if base 10 were to be formed by more than one piece, the pieces could be attached by any means, including, but not limited to, fasteners, adhesives, ultrasonic welding, male/female mating members, or combinations thereof.

Soft pad portion or nasal pillow 200 is attached to base 10, so that the lower border 224 of soft pad portion 200 fits within the space or channel 63 between exterior wall 60 and interior wall 61 of base 10. Structure to enhance the frictional engagement between border 224 and channel 61, such as raised features (e.g. longitudinal ribs 67 extending radially from the interior wall 61 of base 10), may be employed if desired to better hold border 224 substantially sealingly in place while still permitting the pad 200 to be removed and replaced.

Hose connectors 180 may be used to attach the fluid supply hose(s) 300 to the base 10, so that fluid can be supplied through the nasal interface into the patient's nostrils. The side entry hoses 300 provide more flexibility and comfort for the patient, as opposed to front entry “trunk” hoses. However, as disclosed previously, one or more front entry hoses may be used in addition to, or in lieu of, the side entry hoses disclosed.

Hose connectors 180 may be held in removable engagement with base 10 via one or more raised annular rib(s) 188 extending from an inner wall of connector 180, which releasably mate with corresponding annular groove 15 defined by the outer walls of inlets 14 and 18 of the base 10. Thus, the hose can be sealingly but removably attached to the nasal interface. Sealing apparatus such as O-rings, gaskets or the like may also be employed to effectuate an extremely airtight sea. However, it has been determined by experimentation that such sealing apparatus need not be employed to provide a surprisingly quiet, airtight seal that can be undone when/if it is desired to separate the hoses from the base 10. In one embodiment, hoses 300 may be connected to connectors 180 by any means, such as overmolding, extrusion, adhesive, other mechanical connection, or the like.

It should be understood that hose connectors such as connectors 180 need not be employed, such that the hoses can be directly connected, removably or permanently, to the base (e.g. at inlets 14, 18). However, it is beneficial to permit the hoses to be removed from the base so that the nasal interface and/or hoses can be cleaned and/or replaced. In one embodiment of connectors 180, the left and right-side hoses are sealingly connected to connectors 180. In one embodiment, connectors 180 define interior passageways which are in turn defined by interior walls that correspond in contour to the shape of the exterior of the hoses. In one embodiment, the interior walls of the connectors define shoulders 182 against which the bitter ends of the hoses abut.

FIG. 8A is a front elevational cross-sectional view of the assembled nasal interface system showing the air flow from the air supply through the pad 200 in accordance with an aspect of the present invention. Referring to FIGS. 1-7 and 8A, fluid flowing in hoses 300 enters base 10 through inlet(s) 14, 18 and continues through airflow passage(s) 12, 16. In the preferred embodiment, the air flow cross-sectional area of passages 12, 16 increases at some point or points prior to reaching outlet(s) areas 32, 42, 72, 82, which causes a reduction in the velocity of the flowing fluid and, concomitantly, reduces the sound that is occasioned by friction with the sidewalls of passages 12, 16 and internal turbulence within the fluid. Those same passages 12, 16 may also employ one or more areas of reduced cross-section to increase the flow of the incoming fluid so as to laminarize the flow, which flow rate may then be reduced by the aforementioned cross-section expansion.

In one embodiment, to reduce sound caused by the flow of fluid through the passages 12, 16, the passages may be curved to gradually and quietly redirect the flow of fluid from the input direction at inlets 14, 18 (essentially parallel to the elongate axes of the hoses 300) toward or nearly toward the opening(s) 216 (and thereby the nares), as shown in FIG. 8A. Doing so maintains the flow of fluid in a laminar state to avoid noise related to turbulent air molecules colliding with interior features of the base and each other.

The extent of the airflow direction change is dependent primarily upon the angle of the incoming fluid in hoses 300 and/or passages 12, 16. The angle between hoses 300 and/or the incoming flow of fluid, on the one hand, and horizontal, on the other, may be dictated by the aesthetic appearance desired. Irrespective of such angle, the inventors have discovered that the gentle redirection of the incoming flow of fluid results in a quieter operation, and thus experience, of the nasal interface. The contour(s) and configuration(s) of the passages within the base 10 may be varied to meet aesthetic and/or functional parameters without departing from the scope of the invention.

In an embodiment, to better maintain the flow of incoming fluid as laminar as possible, and to better distribute the transfer of energy from the air molecules to the base, redirection vanes such as vanes 22, 24 may optionally be employed. Vanes 22, 24 cause a portion of the incoming fluid to be redirected, while the central inlet passageway walls 15, 17 redirect the remainder of the incoming fluid. Additional vanes may be employed to distribute the load incurred by the vanes and the walls 15, 17 even further. The vanes positioned within passages 12, 16 take some of the energy associated with the redirection of the flowing air so that passage sidewalls 15, 17 don't have to handle all of the energy transfer, resulting in reduced noise.

The trailing edges 23, 25 of vanes 22, 24, respectively, are preferably oriented so that the direction of the fluid flow departing from the vanes is essentially toward, or nearly toward, the openings 216 to further enhance the low-noise characteristics, and comfort, of the nasal interface.

Central inlet passageway walls 15, 17, in an embodiment, are spaced apart to define an exhaust passage 100 through which excess, exhaled or other expendable fluid may be expelled. Inhaled air may also be taken in through exhaust passage 100. Passage 100 may be shaped so as to gradually and quietly redirect the flow of expelled (or intake) fluid from the upper end of passage 100 to the direction of the opening(s) 102, 104 (and thereby the ambient), as shown in FIG. 8B. Doing so likewise maintains the flow of fluid in a laminar state to avoid noise related to turbulent air molecules colliding with interior features of the base and each other.

In an embodiment, the exhaust passages 110, 113 as well as exhaust outlets 102, 104 direct expelled fluid toward the side and not the front of the base. This will benefit the bed partners of users by permitting the user to directly face the partner without exhaled air being directed at the partner.

Fluid enters the interior of the pillow 200 through the area surrounded by the lower border 224. The fluid continues through the interior of the pillow 200 and enters the patient's nostrils through hole(s) 216. To the extent that the fluid inside pillow 200 exceeds the ambient pressure external to the pillow, the upper wall(s) 210 of the pillow are caused to press against the underside of the wearer's nose, creating an effective (and quieting) seal.

FIG. 8B is a front elevational cross-sectional view of the assembled nasal interface system showing the expelled air flow through the pillow and subsequently out through the base in accordance with an embodiment of the present invention. Referring to FIGS. 1-7 and 8B, fluid can be expelled through holes 216 defined by the upper wall(s) 210 of the pillow 200. The fluid then exits the pillow 200 through the interior of the lower border 224 into base 10, then being forced out of exhaust passages 12, 14 and outlets 14, 16. Excess fluid may also build up in the base 10 when the patient is not inhaling, which is vented through passages 110, 112. In addition, the patient may breathe through the vent passages 110, 112 in the event that the air supply is inoperable.

Exhaust passage 100, in an embodiment, may be shaped so as to decrease in cross-section until fluid flow is choked at an area of reduced cross-section 106 to create a laminar flow, and then the exhaust passage expands to slow down and quiet the airflow. In this embodiment, divider 140 then splits the fluid flow into vent airflow passages 110, 112. In the preferred, but not only, embodiment, vent airflow passages 110, 112 increase in cross-sectional area until the fluid is finally expelled at vent outlet(s) 102, 104.

As stated previously, vent outlets 102, 104 are preferably positioned to be directed obliquely to the patient's face so that the air is not expelled onto the patient or on somebody that may be in close proximity to the patient.

In an embodiment, the invention is directed to a nasal interface base for use in connection with a CPAP system, the CPAP system including a pressurized air supply unit, a nasal interface, and first and second air supply hoses adapted to supply pressurized air supplied by the pressurized air supply unit to the interface, the interface including a base member and a resilient nasal pillow, the pillow including a lower border adapted to releasably mate with the base, the improvement comprising: the base defining connection structure for releasably connecting the lower border of the pillow to the base; the base also defining first and second airflow inlets leading to respective first and second airflow channels, the channels fluidly communicating the respective first and second air supply hoses with a hollow interior of the base; the left and right channels each being positioned within the base to receive a flow of pressurized air from respective left and right hoses, and to redirect the flow from a first direction parallel to respective sidewalls of the left and right hoses to a second direction which is not parallel to the first direction.

The nasal interface base can further comprise a bifurcated exhaust passage fluidly communicating the hollow interior of the base with the ambient.

The nasal interface base can further comprise first and second exhaust outlets fluidly communicating the bifurcated exhaust passage with the ambient, the first and second exhaust passages oriented in such a way that they do not result in expired air being directed toward a bed partner who is positioned in face-to-face relationship with a wearer of the nasal interface.

In another embodiment, the invention is directed to a nasal interface system, comprising: apparatus for supplying pressurized air; a nasal pillow adapted to be placed in substantially sealing engagement with the underside of a wearer's nose, the nasal pillow defining a substantially hollow interior; a base comprising a partially hollow and interior left and right airflow inlets fluidly communicated with respective left and right airflow passages; first and second hoses for directing the pressurized air to the left and right airflow inlets, respectively, the left and right airflow inlets and left and right airflow passages fluidly communicating the pressurized air with the interior of the base; the left and right airflow passages defined by respective sidewalls which are gently curved so as to redirect the pressurized air from first respective directions to second respective directions, the second respective directions being generally toward the hollow interior of the nasal pillow.

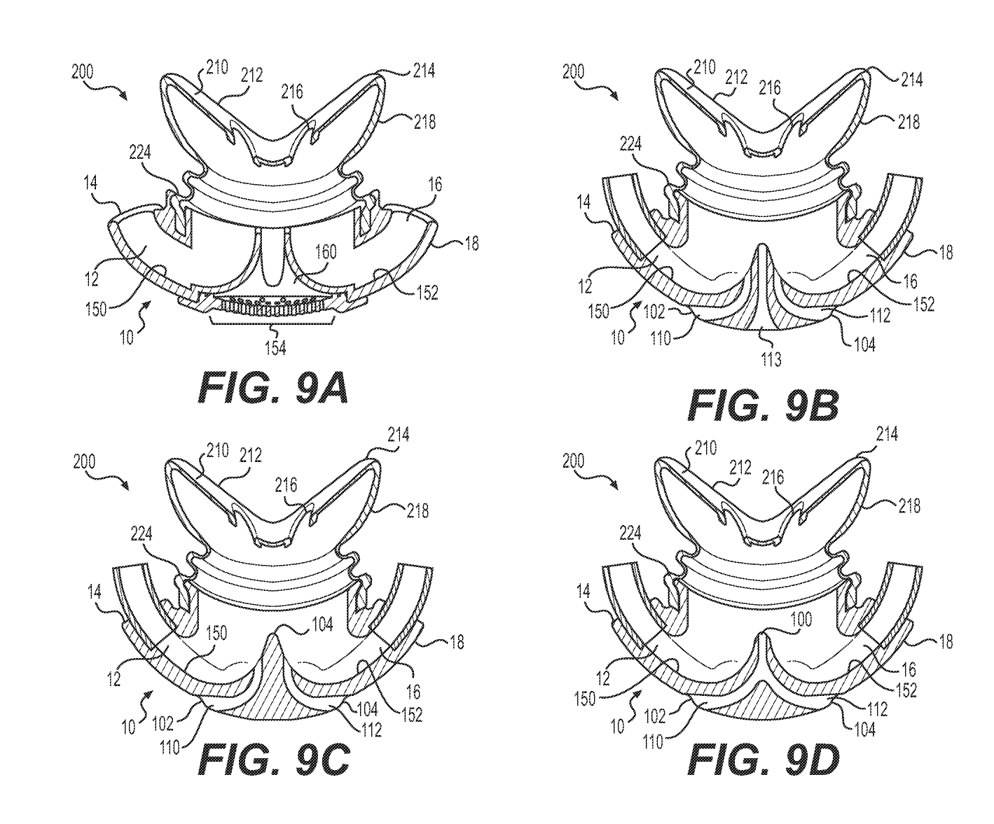

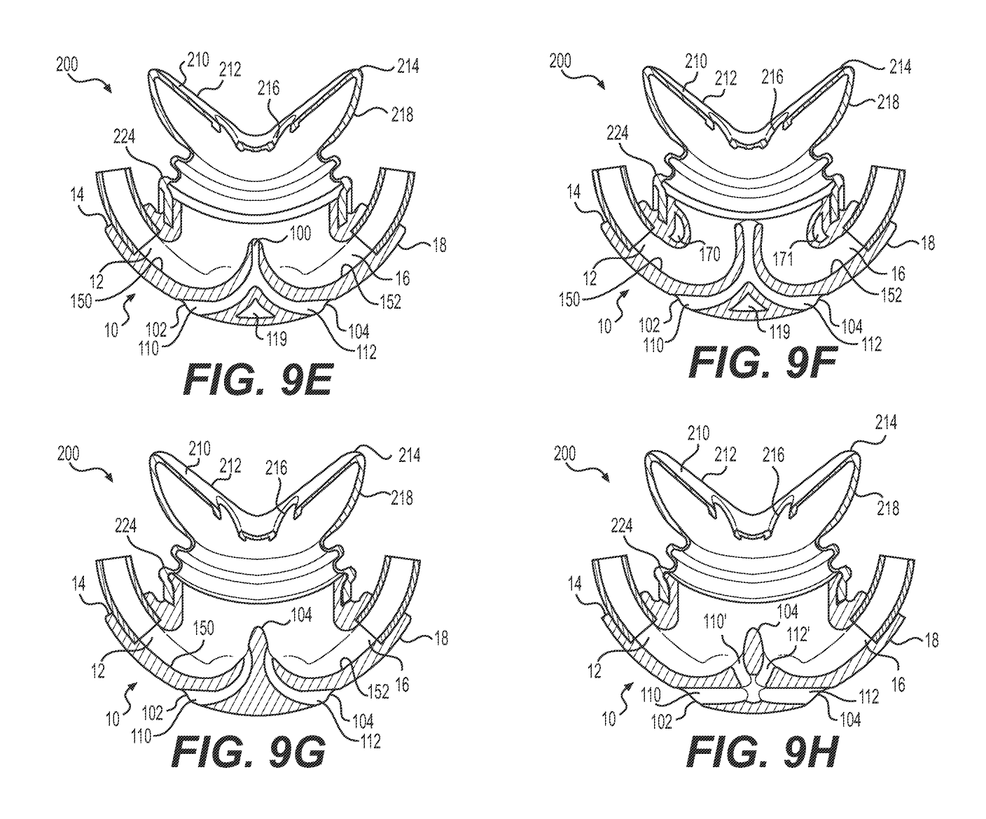

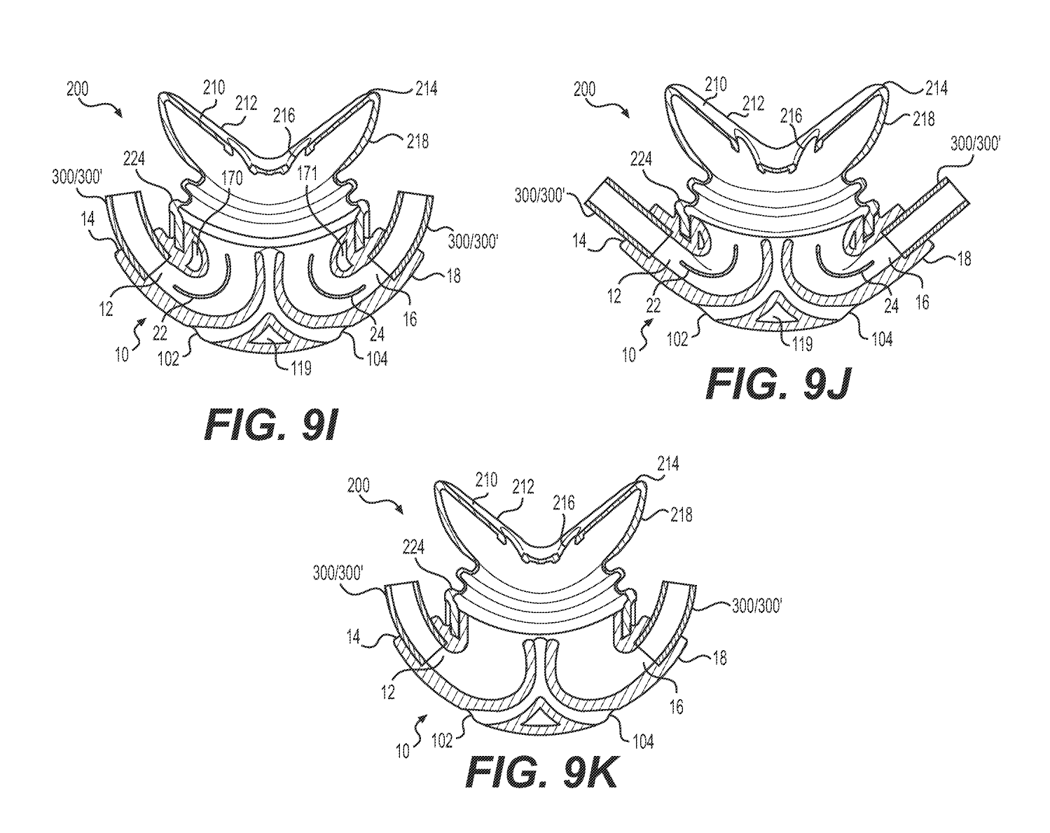

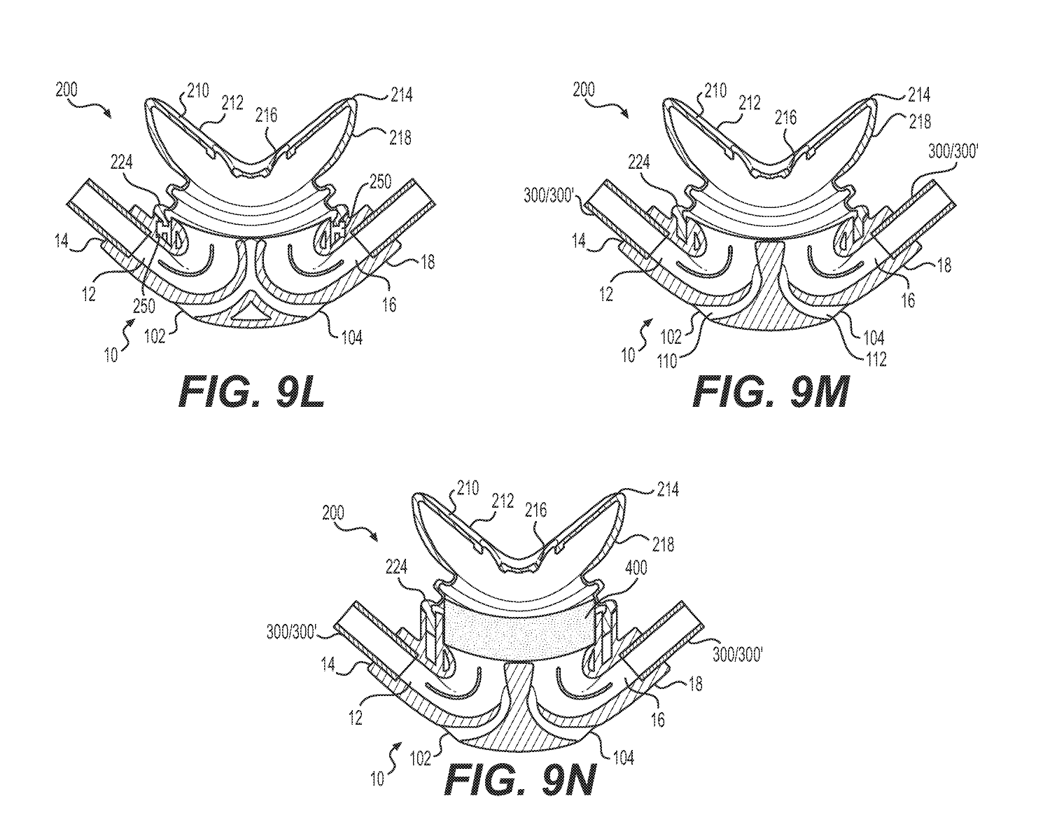

As shown by the embodiments in FIGS. 9A-9N, the nasal interface base 10 (and additionally in certain embodiments nasal pillow 200) can be formed in different shapes and sizes, with differing interior and exterior features/contours, to provide a sleek, comfortable, and quiet patient interface that provides the least resistance to the patient's movement, a pleasant ornamental appearance, while still maintaining a substantially leak proof seal between the patient interface and the face of the patient.

FIGS. 9A-9N illustrate a variety of alternative, exemplary, inner structural arrangements which the base 10 may be configured in. These embodiments are merely examples of the wide variety of structural features which can be employed in carrying out the novel principals of the invention.

FIG. 9A shows a base 10 with a nasal pillow 200 removably attached thereto in a manner similar to that described in connection with FIGS. 1 through 8. Air passageways 12, 16 are defined in part by curving walls 150, 152, respectively, which gently change the direction of flow of air flowing there through so as to permit the redirection of the flowing air toward the openings 216 while keeping the level of turbulence, and hence unwanted sound, to a minimum. Passageways 12, 16 fluidly communicate fluid openings 14, 16 with the interior of nasal pillow 200. This configuration, as well as all of the configurations disclosed elsewhere herein, permit side-entry airflow while gently and quietly redirecting the flow of fluid/air toward the nasal pillow 200.

Expired air can be expelled out of base 10 in any manner desired. Expired air is able to flow into exhaust passage 160. As shown in FIG. 9, a diffuser or the like 154 can be employed to diffuse expired air. As is obvious to the skilled artisan, air or other fluids may be taken in through passageway 160 and into the interior of nasal pillow 200 as well.

FIG. 9B shows a slightly modified form of the base shown in FIG. 9, wherein a plurality of discrete exhaust passages 110, 112 and 113 are employed. Any number of such passages may be employed for the purposes desired. As can be seen, passages 110, 112 and 113 have gently sloping sides to keep noise creation to an absolute minimum.

FIG. 9C shows an embodiment similar to that shown in FIG. 9B, but where central passageway 113 is omitted, so that expired air is not directed toward a nearby bed partner.

FIG. 9D discloses in embodiment similar to FIG. 9C, but where exhaust passageways 110, 112 diverge from a common passageway 100 adjacent the interiors of base 10 and nasal pillow 200.

FIG. 9E discloses the embodiment of FIG. 9D, but where a void portion 119 of base 10 is removed to save on the utilization of material, saving cost and weight. FIG. 9F shows the same embodiment as disclosed in FIG. 9E, but where additional material voids 170, 171 are used for the purpose of material and weight reduction. Any number, manner or size of such voids may be employed as will occur to those of skill in the art.

FIG. 9G discloses a base 10 having a slightly modified septum 104, which has a bulbus configuration and results in entrances 113, 115 to passageways 110, 112, respectively, which are oriented at oblique angles relative to the flow of air passing from the interior of nasal pillow 200 toward passageways 110, 112, to further aid in noise attenuation.

FIG. 9H shows a base 10 having a further revised septum 104, which creates exhaust passage inlets 110′, 112′ which lead to exhaust passages 110, 112. Exhaust passages 110, 112 may be joined as a common passageway as shown in the figure, or a wall may be positioned there between, such that exhaust passage inlet 110′ leads only to exhaust passage 110, and exhaust inlet 112′ leads only to exhaust passage 112.

FIG. 9 discloses an embodiment of a nasal interface base 10 similar to that shown in FIG. 8, but with material voids 170, 171 associated there with. In addition, the angle of entry of fluid supply tubes 300/300′ is slightly different in this embodiment to demonstrate the wide variety of orientations of the sound attenuation and aesthetic styling which may be incorporated into the invention.

FIG. 9J discloses a base 10 similar to that shown in FIGS. 1-8, but which has a somewhat lower profile, making it smaller and even more comfortable than certain other embodiments disclosed herein. A smaller nasal interface base and/or nasal pillow may be employed for smaller wearers such as children.

FIG. 9K is similar to the embodiment shown in FIG. 9F, but where the material voids 170, 171 are not employed.

FIG. 9L shows an embodiment of a nasal interface base 10 similar to that shown in FIGS. 1-8, but further includes a snap ring 250 adapted to releasably join lower border 224 of nasal pillow 200 with base 10. Connection means such as snap ring 250 permits a relatively secure, positive connection between pillow 200 and base 10 while at the same time permitting ready separation thereof.

FIG. 9M discloses in embodiment similar to FIG. 9L, but where septum 104 is shaped somewhat differently than that used in other environments disclosed herein, which separates exhaust passages 110, 112 from each other and provides desired sound attenuation.

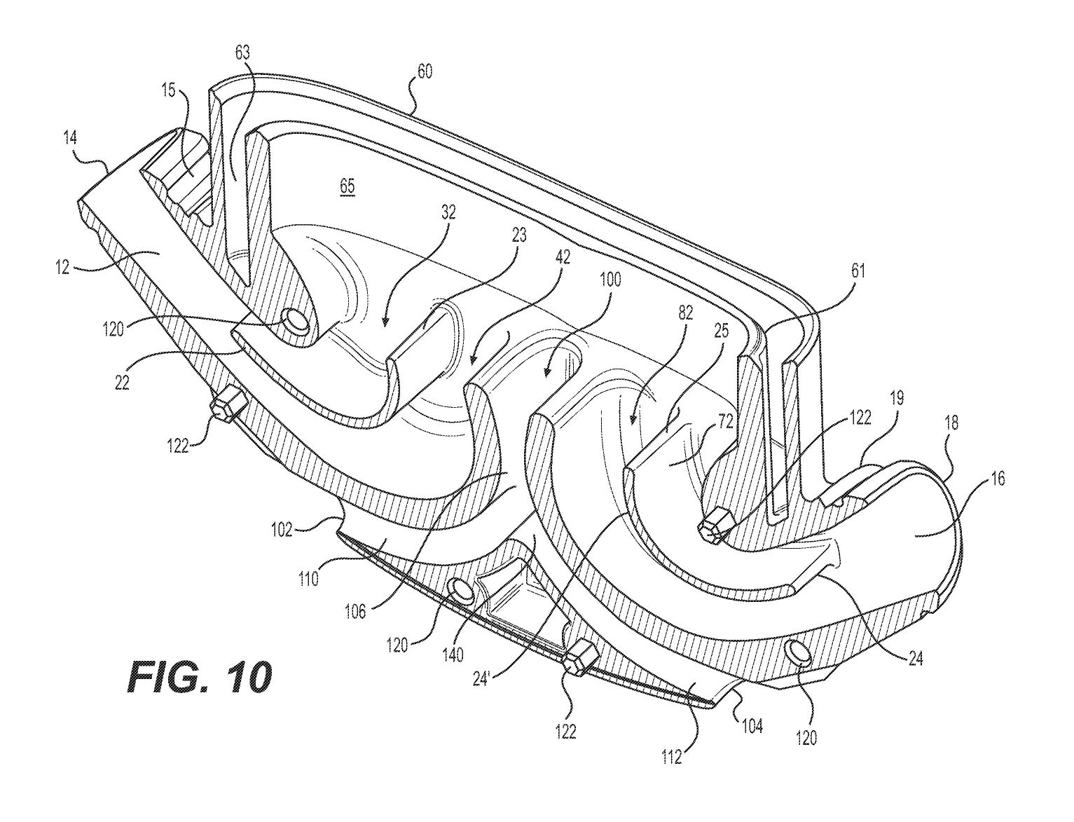

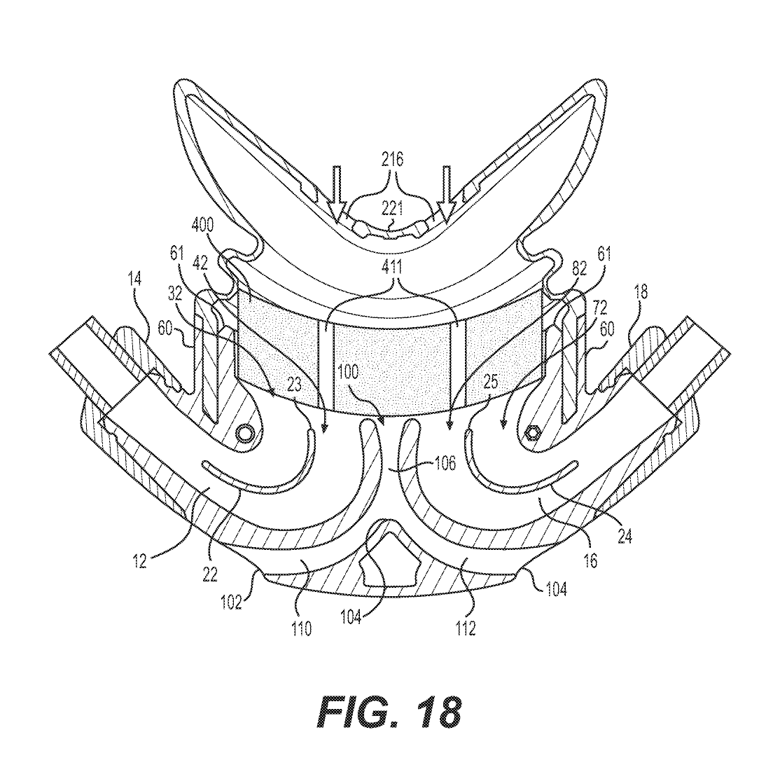

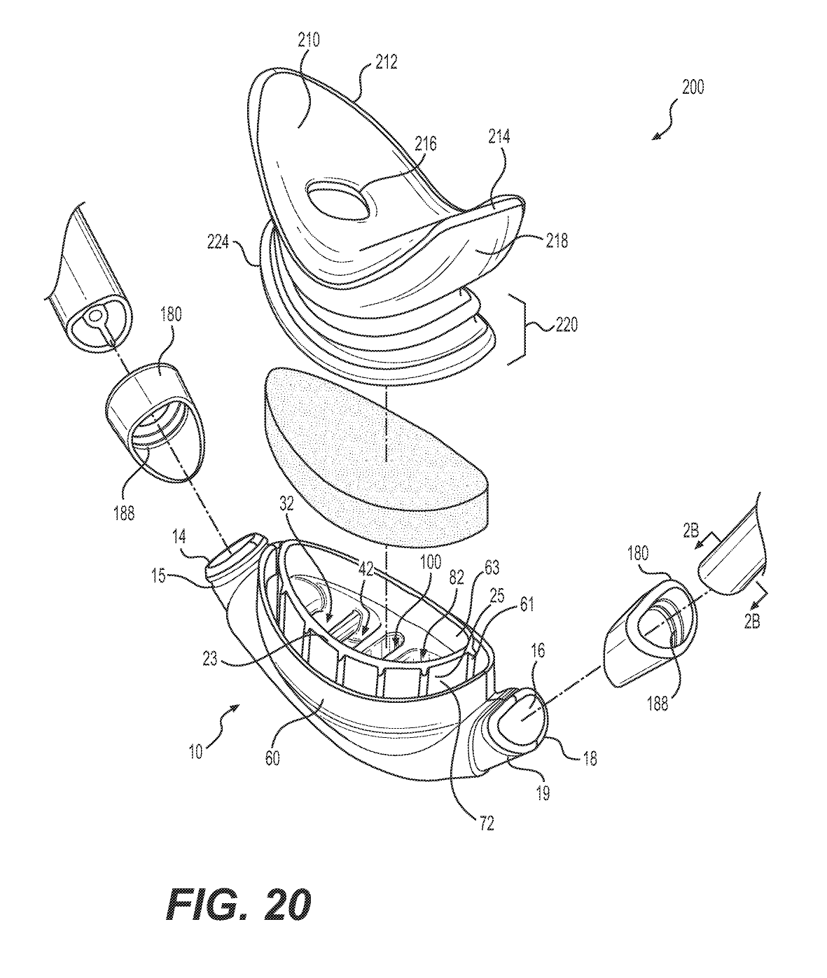

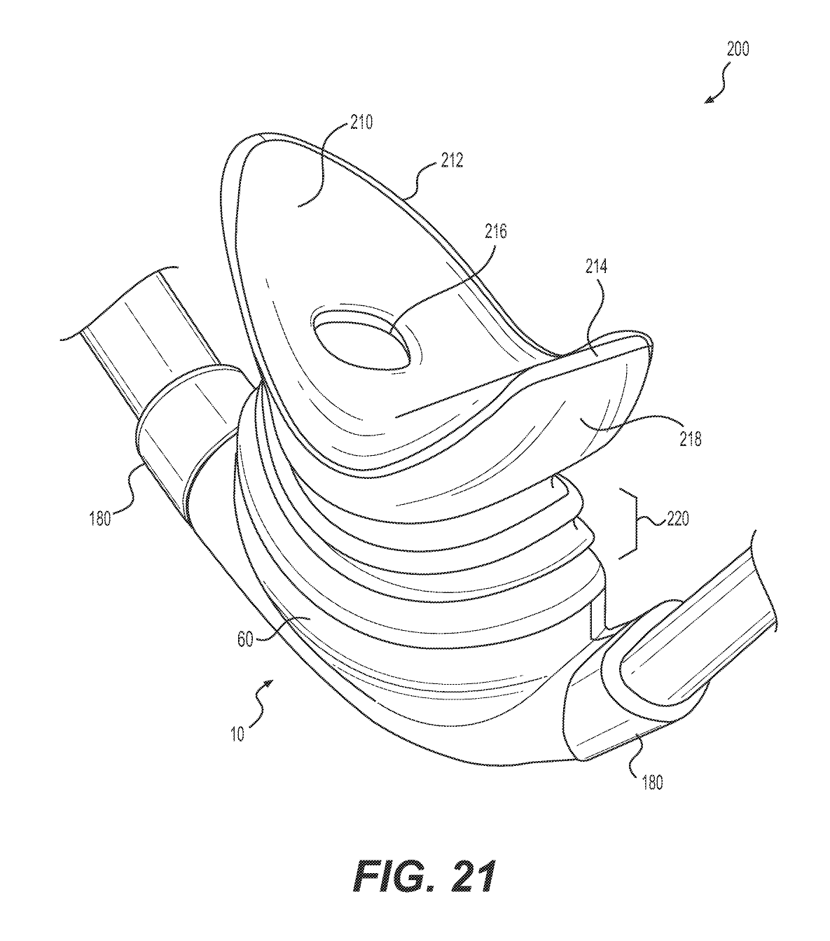

FIG. 9N discloses a base 10 in accordance with the embodiments shown in FIGS. 10-21, wherein a humidification and/or sound attenuation element 400 is employed in the manner disclosed elsewhere herein.

It is to be appreciated that the fluid flow hoses 300/300′, and the connection thereof to the various bases 10 disclosed herein, may be of any orientation, including those that are non-circular in cross-section. In addition, connectors 180 need not be used to connect hoses 300/300′ to any of the bases 10. Rather, the hoses may be integrally molded with the bases 10, or may simply be inserted therein, removably or permanently. Alternatively, any arrangement for removably connecting suitable fluid supply hoses to any of the bases 10 disclosed herein are considered to be within the scope of the invention.

Alternatively, as shown in FIGS. 10-18, exterior wall 60 and interior wall 61 can be elongated (i.e., raised) to accommodate sound-deadening and/or humidification elements/material 400, such as Humidification Moisture Exchanger (“HME”) element or sponge, within space 65 defined by interior wall 61 and/or other features of the base 10. The element 400 could be located beneath and/or surrounding lower border 224 of soft pad portion 200. Any material can be used in order to act as a sound attenuator to absorb the acoustic vibrations caused by the nasal interface or the patient.

In an embodiment of the invention, the sound-deadening and/or humidification elements/material 400 may include an HME or an integrated HME muffler. The HME or integrated HME muffler is preferably removably positioned within the volume 65 defined by base 10. The HME can be made out of foam, a pleated filter, paper, or any substance which acts as a condensation absorption media. The integrated HME muffler can be made out of any material that acts as a condensation absorption media, as well as any material that can absorb acoustic vibrations. The HME or integrated HME muffler may be placed anywhere that would allow the user to obtain the humidification or acoustic absorption benefits of the material. However, placing the HME or integrated HME muffler within base 10 or pillow 200 realizes the benefit of capturing the greatest amount of moisture due to its close proximity to the nares of the wearer.

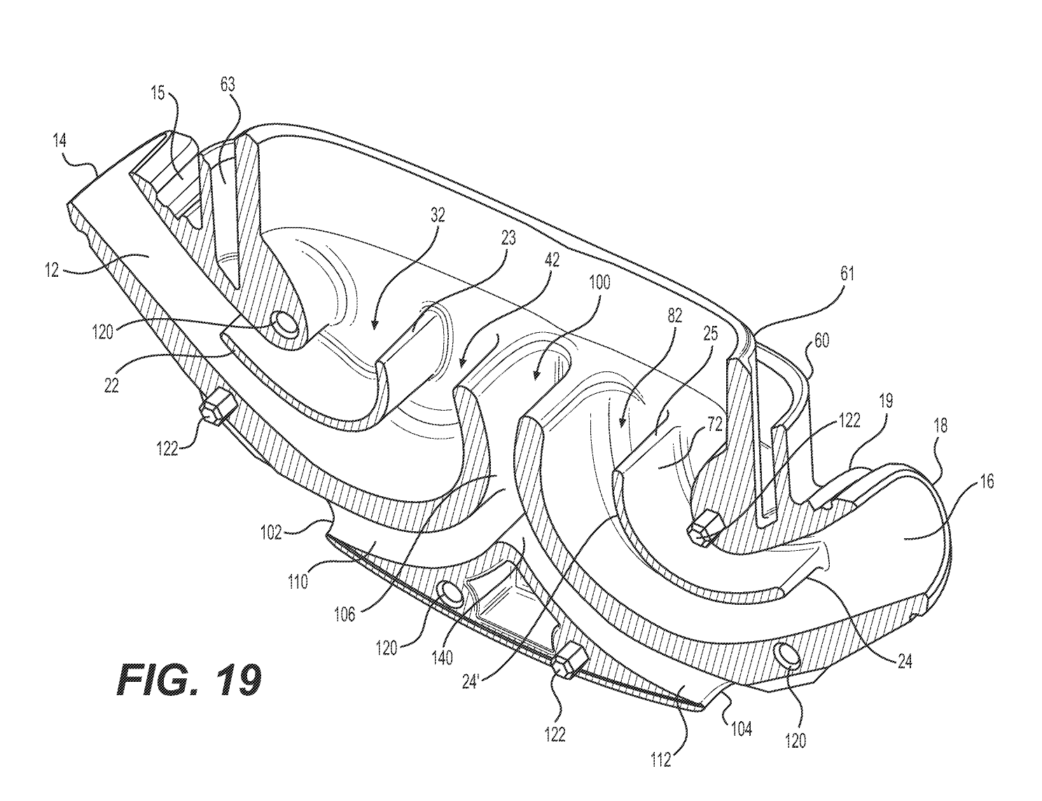

Alternatively, as shown in FIGS. 19-21, exterior wall 60 need not be elongated (i.e., raised) to accommodate sound-deadening and/or humidification elements/material 400 within space 65 and/or other features of the base 10. The element 400 can be located beneath and/or surrounding lower border 224 of soft pad portion 200. Lower border 224 of pillow 200 can still be removably retained in the space 63 defined by outer and inner walls 60, 61, respectively, even if outer wall 60 is not raised. Keeping outer wall 60 shorter carries with it the benefit of maintaining a lower profile (i.e., height off of face) of the nasal interface, because of the shorter moment arm presented by the interface.

It is to be appreciated that the element 400 can be located anywhere within the base or pillow, or both, without departing from the spirit of the invention. However, locating the element 400 prior to the leakage opening/vent passage (such as passage 100) captures the greatest amount of moisture possible, unlike any prior system.

It is also to be appreciated that the combination of the gently curving redirection walls and the exhaust vents, in the embodiments in which both are present, provides the ideal noise solution by permitting the proper amount of leakage flow to the exterior of the base 10.

For the purpose of explanation, various features and embodiments of a breathing apparatus and/or elements of a breathing apparatus have been described and depicted in the figures. It should be appreciated that the various features and embodiments may be susceptible to combination and substitution. For example, various features shown and/or described relative to one or more embodiments may be combined with features shown and/or described relative to one or more other embodiments. Similarly, features described and/or shown relative to one or more embodiments may be substituted with features described and/or shown relative one or more other embodiments.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable those of ordinary skill in the art to understand the inventions in their various embodiments with various modifications as are suited to the particular use contemplated.

Benefits, other advantages, and solutions to problems have been described above with regard to specific embodiments of the present inventions. However, the benefits, advantages, solutions to problems, and any element(s) that may cause or result in such benefits, advantages, or solutions to become more pronounced, are not to be construed as a critical, required, or essential feature or elements of any or all of the claims. The invention is defined solely by the appended claims including any amendments made while this application is pending, and all equivalents of those claims as issued.

What is claimed is:

1. In a nasal interface base for use in connection with a CPAP system, the CPAP system including a pressurized air supply unit, a nasal interface, and first and second air supply hoses adapted to supply pressurized air supplied by the pressurized air supply unit to the interface, the interface including a base member and a resilient nasal pillow, the pillow including a lower border adapted to releasably mate with the base, the improvement comprising:
the base defining connection structure for releasably connecting the lower border of the pillow to the base;
the base also defining left and right airflow inlets leading to respective left and right airflow channels, the channels fluidly communicating the respective first and second air supply hoses with a hollow interior of the base;
the left and right channels each being positioned within the base to receive a flow of pressurized air from respective first and second hoses, and to redirect the flow from a first direction parallel to respective sidewalls of the first and second hoses to a second direction which is not parallel to the first direction.

2. The nasal interface base of claim 1, further comprising:
a bifurcated exhaust passage fluidly communicating the hollow interior of the base with the ambient.

3. The nasal interface base of claim 2, further comprising:
left and right exhaust outlets fluidly communicating the bifurcated exhaust passage with the ambient, the left and right exhaust passages oriented in such a way that they do not result in expired air being directed toward a bed partner who is positioned in face-to-face relationship with a wearer of the nasal interface.

4. The nasal interface base of claim 1, further comprising:
a sound damping element disposed within at least a portion of the hollow interior of the base.

5. The nasal interface base of claim 1, further comprising:
a humidification element disposed within at least a portion of the hollow interior of the base.

6. The nasal interface base of claim 5, wherein the humidification element is a humidification moisture exchanger.

7. The nasal interface base of claim 1, further comprising left and right hose connectors adapted to releasably connect the left and right hoses to the respective first and second airflow inlets.

8. The nasal interface base of claim 7, wherein the left and right hose connectors are overmolded with the first and second hoses, the left and right hose connectors each defining connection structure adapted to mate with corresponding structure defined by the first and second airflow inlets to permit a snap connection therebetween.

8. A nasal interface system, comprising:
apparatus for supplying pressurized air;
a nasal pillow adapted to be placed in substantially sealing engagement with the underside of a wearer's nose, the nasal pillow defining a substantially hollow interior;
a base comprising a partially hollow and interior left and right airflow inlets fluidly communicated with respective left and right airflow passages,
first and second hoses for directing the pressurized air to the left and right airflow inlets, respectively, the left and right airflow inlets and left and right airflow passages fluidly communicating the pressurized air with the interior of the base;
the left and right airflow passages defined by respective sidewalls which are gently curved so as to redirect the pressurized air from first respective directions to second respective directions, the second respective directions being generally toward the hollow interior of the nasal pillow.

9. The nasal interface base of claim 1, further comprising:
a bifurcated exhaust passage fluidly communicating the hollow interior of the base with the ambient.

10. The nasal interface base of claim 2, further comprising:
left and right exhaust outlets fluidly communicating the bifurcated exhaust passage with the ambient, the left and right exhaust passages oriented in such a way that they do not result in expired air being directed toward a bed partner who is positioned in face-to-face relationship with a wearer of the nasal interface.

11. The nasal interface base of claim 1, further comprising:
a sound damping element disposed within at least a portion of the hollow interior of the base.

12. The nasal interface base of claim 1, further comprising:
a humidification element disposed within at least a portion of the hollow interior of the base.

13. The nasal interface base of claim 5, wherein the humidification element is a humidification moisture exchanger.

14. The nasal interface base of claim 1, further comprising left and right hose connectors adapted to releasably connect the left and right hoses to the respective first and second airflow inlets.

15. The nasal interface base of claim 7, wherein the left and right hose connectors are overmolded with the first and second hoses, the left and right hose connectors each defining connection structure adapted to mate with corresponding structure defined by the first and second airflow inlets to permit a snap connection therebetween curving walls which divert a flow of air flowing from the
